(19) United States
(12) Patent Application Publication
(10) Pub. No.: US 2007/0134678 A1
(43) Pub. Date: Jun. 14, 2007

(54) Comparative genome hybridization of organelle genomes

(76) Inventor: Dianne M. Rees, Sunnyvale, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/302,016

(22) Filed: Dec. 12, 2005

(51) Int. Cl. C12Q 1/68; C12M 3/00

(52) U.S. Cl. 435/6; 435/287.2

(57) ABSTRACT

A comparative genome hybridization method is provided. In certain embodiments, the method comprises: a) contacting a first array of surface-bound nucleic acid probes with: i) a first labeled population of target nucleic acids from a genome of an organelle of a first cell; and ii) a second labeled population of target nucleic acids from a genome of an organelle of a second cell; and b) detecting binding between said surface-bound nucleic acid probes and the first and second labeled populations of target nucleic acids. Compositions for use in the subject methods are also provided.

### BACKGROUND

Mitochondria and plastids are organelles that contain a genome.

Nearly all eukaryotic cells contain multiple mitochondria and each mitochondrion within a eukaryotic cell contains multiple copies of a circular DNA genome. The mitochondrial genome is distinct from the nuclear genome and is contained within the mitochondrion. More than 5000 copies of the mitochondrial genome may be present within a single eukaryotic cell. Mitochondria manufacture adenosine triphosphate (ATP) by oxidative phosphorylation.

Plastids are found in plant cells and the cells of certain photosynthetic algae. Plastids are responsible for photosynthesis, storage of products like starch and oils, and the synthesis of many classes of molecules such as fatty acids, terpenes and pigments. Like mitochondria, plastids have their own genome—the plastid genome. Also like mitochondria, a plastid may contain many copies (e.g., from about 20 to about 100, or more copies) of a plastid genome and a cell may contain many plastids.

Methods for analysis of organelle genomes, e.g., the genomes of mitochondria and plastids, are provided.

### SUMMARY OF THE INVENTION

A comparative genome hybridization method is provided. In certain embodiments, the method comprises: a) contacting a first array of surface-bound nucleic acid probes with: i) a first labeled population of target nucleic acids from a genome of an organelle of a first cell; and ii) a second labeled population of target nucleic acids from a genome of an organelle of a second cell; and b) detecting binding between the surface-bound nucleic acid probes and the first and second labeled populations of target nucleic acids. Compositions for use in the subject methods are also provided.

In certain embodiments, the method may comprise determining relative amounts of bound first labeled populations of target nucleic acids and bound second labeled populations of target nucleic acids.

In certain embodiments, the method may include determining differences in numbers of copies of one or more sequences in organelle genomes of the first and second cells.

In certain embodiments, the method may include determining the relative expression of one or more nuclear encoded gene products in the first and second cells.

In certain embodiments, the method may include determining differences in numbers of copies of one or more sequences in nuclear genomes of the first and second cells.

In certain embodiments, the method may employ a multi-array substrate.

In certain embodiments, the method may include correlating relative amounts determined with a condition of an organism from which the first or second cell was obtained. The condition may be a mitochondrial-related disease, e.g., a muscle-related, hearing-related or vision-related disorder.

In certain embodiments, the method may include correlating relative amounts determined with an amount of heteroplasmy.

In certain embodiments, the first cell and the second cell are obtained from different tissues of the same patient and the method evaluates differences in numbers of copies of one or more sequences in organelle genomes of the different tissues.

In certain embodiments the first cell and the second cell are obtained from the same patient at different times and the method evaluates differences in numbers of copies of one or more sequences in organelle genomes at the different times.

In certain embodiments at least one of the first and second cells is exposed to a stimulus and the method evaluates differences in numbers of copies of one or more sequences in organelle genomes in response to the stimulus. The stimulus may be a chemical compound such as a drug or an environmental condition.

In certain embodiments, the results may be employed to evaluate the identity of an organism from which the first or second cell was obtained.

In certain embodiments, the surface-bound nucleic acid probes may be for detecting a plurality of different organelle genome regions.

In certain embodiments, the first and second labeled populations of target nucleic acids are distinguishably labeled.

In certain embodiments, the first and second cells may be, for example, cultured cells or cells obtained from a subject.

In certain embodiments, the method may be employed to identify deletions or insertions in the genome of the organelle of the first cell relative to the genome of the organelle of the second cell.

In certain cases, detecting produces a first data set that may be stored.

In certain embodiments, the method may include: c) contacting a second array of surface-bound nucleic acid probes with: i) a third labeled population of target nucleic acids from a nuclear genome of the first cell; i) a fourth labeled population of target nucleic acid from a nuclear genome of the second cell; and d) detecting binding between the surface-bound polynucleotide probes and the third and fourth labeled populations of polynucleotides. In this method, the first array and the second array may be on the same substrate or on different substrates. A second data set may be produced using this method.

In certain embodiments, the organelle may be a chloroplast or mitochondria, for example.

In another embodiment, a multi-array substrate comprising: a) a first array of surface-bound nucleic acid probes that bind to regions of an organelle genome of a cell; and b) a second array of surface-bound nucleic acid probes that bind to regions of a nuclear genome of the cell, wherein the first array and the second array are spatially separated, is provided. The surface-bound nucleic acid probes may be oligonucleotide probes.

In certain embodiments, the first array may contain surface-bound nucleic acid probes for detecting intergenic regions of the organelle genome.

In certain embodiments, the organelle genome may be a mitochondrial genome and the cell may be an animal cell.

In certain embodiments, the organelle genome may be a chloroplast genome and the cell may be a plant cell.

In another embodiment, a kit comprising the above-described multi-array substrate and reagents for obtaining genomic DNA from an organelle of a eukaryotic cell are provided. The kit may optionally include reagents for labeling the genomic DNA.

### Definitions

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 2 to about 200 nucleotides in length (e.g., about 10 to about 100 nucleotides or about 30 to about 80 nucleotides) while a “polynucleotide” or “nucleic acid” includes a nucleotide multimer having any number of nucleotides. Oligonucleotides may be synthetic A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in certain embodiments, known. An array feature is generally homogenous in composition and concentration and the features may be separated by intervening spaces (although arrays without such separation can be fabricated).

The term “substrate” as used herein refers to a surface upon which probes, e.g., an array, may be adhered. Substrates may be porous or non-porous, planar or non-planar over all or a portion of their surface. Glass slides are the most common substrate for arrays, although fused silica, silicon, plastic and other materials are also suitable. A substrate may contain more than one array.

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. The support can be planar, nonplanar or a combination thereof. The support can be porous or non-porous. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays. “Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

In certain embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO4, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

Wash conditions used to remove unbound nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The term “highly stringent hybridization conditions” as used herein refers to conditions that are compatible to produce complexes between complementary binding members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array).

Additional hybridization methods are described in references describing CGH techniques (Kallioniemi et al., Science 1992;258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol. 1981 ;21:470-480 and Angerer et al., In Genetic Engineering: Principles and Methods, Setlow and Hollaender, Eds. Vol 7, pgs 43-65 (Plenum Press, New York 1985). See also U.S. Pat. Nos.: 6,335,167; 6,197,501; 5,830,645; and 5,665,549; the disclosures of which are herein incorporated by reference.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human nuclear genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed. As used herein, a “reference nucleic acid sample” or “reference nucleic acids” refers to nucleic acids comprising sequences whose quantity or degree of representation (e.g., copy number) or sequence identity is known.

Similarly, “reference genomic acids” or a “reference genomic sample” refers to genomic nucleic acids comprising sequences whose quantity or degree of representation (e.g., copy number) or sequence identity is to be compared with a test nucleic acids. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

If a surface-bound polynucleotide or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

“CGH array” and “aCGH array” refer to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein and thus can also be referred to as a “location analysis array” or an “array for ChIP-chip analysis.” In certain aspects, a CGH array provides probes for screening or scanning a genome of an organism and comprises probes from a plurality of regions of the genome.

Generally, the instant method includes determining relative differences in copy number and does not encompass determining single nucleotide polymorphisms (SNPs) between sequences. However, in certain aspects, the method can be used to detect microdeletions or microamplifications, loss or gain of one or a few bases. However, generally, as used herein, a “duplication” or “amplification” of a genomic sequence refers to the addition of more than a single base to the genomic sequence as compared to a reference sequence. Similarly, a “deletion” of a genomic sequence generally refers to the loss of more than a single base of a genomic sequence as compared to a reference sequence.

In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

In certain aspects, in constructing arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences and such sequences can, optionally, be all non-transcribed sequences (e.g., intergenic regions including regulatory sequences such as promoters and/or enhancers lying outside of transcribed regions).

In certain aspects, an array may be optimized for one type of genome scanning application compared to another, for example, the array can be enriched for intergenic regions compared to coding regions for a location analysis application. In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic regions (e.g., non-coding regions which exclude introns and untranslated regions, i.e., comprise non-transcribed sequences) of a nucleotide sample of interest.

In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular genomic region with certain disease conditions. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for scanning a nuclear genome in addition to scanning an organelle genome. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome). Additional chromosomal imbalances that may be scanned include, but are not limited to those described in WO 93/18186, the entirety of which is incorporated by reference.

“Themed” arrays may be fabricated, for example, arrays including whose duplications or deletions are associated with specific types of pathologies. The selection of such arrays may be based on patient information such as familial inheritance of particular genetic abnormalities or a predisposition or risk of having an organelle-associated disease. In certain aspects, an array for scanning an entire genome is first contacted with a sample and then a higher-resolution array is selected based on the results of such scanning.

Themed arrays also can be fabricated for use in gene expression assays, for example, to detect expression of genes involved in selected pathways of interest, or genes associated with particular diseases of interest.

In one embodiment, a plurality of probes on the array are selected to have a duplex Tₘ within a predetermined range. For example, in one aspect, at least about 50% of the probes have a duplex Tₘ within a temperature range of about 75° C. to about 85° C. In one embodiment, at least 80% of the polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about I.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In still other aspects, probes on the array comprise at least coding sequences. In one aspect, probes represent sequences from an organism such as Drosophila melanogaster, Caenorhabditis elegans, yeast, fish (e.g., such as a zebrafish), a bird, a mouse, a rat, a domestic animal, a companion animal, a primate, a human, etc. In certain aspects, probes representing sequences from different organisms are provided on a single substrate, e.g., on a plurality of different arrays.

A “CGH assay” using an aCGH array can be performed as follows, in certain embodiments. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having a disease associated with a defect in an organelle genome (e.g., within specific genes within an organelle genome or associated with abnormal levels of normal or variant organelle genomes) and/or a disease associated with a defect in a nuclear genome which results in abnormal levels of normal or variant organelle genomes.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, the reference target molecules in a population are present at a level comparable to an average level found in one or more individuals without a disease associated with a defect in an organelle genome (e.g., within specific genes within an organelle genome or associated with abnormal levels of normal or variant organelle genomes) and/or without a disease associated with a defect in a nuclear genome which results in abnormal levels of normal or variant organelle genomes. The relative proportions of complexes formed labeled with the first label vs. the second label can be used to evaluate relative copy numbers of targets found in the two samples.

In certain aspects, test and reference populations of nucleic acids may be applied separately to separate but identical arrays (e.g., having identical probe molecules) and the signals from each array can be compared to determine relative copy numbers of the nucleic acids in the test and reference populations.

Methods to fabricate arrays are described in detail in U.S. Pat. Nos. 6,242,266; 6,232,072; 6,180,351; 6,171,797 and 6,323,043. As already mentioned, these references are incorporated herein by reference. Drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

A “multi-array substrate” is a substrate that contains more than one array (e.g., 2, 4, 6, 8, 10, 12, 16, 24 or more than 24 arrays, etc.) disposed upon a surface of the substrate. Any number or all of the arrays may be the same as or different from one another. The arrays of a multi-array substrate are generally spatially separate. The distance between the neighboring arrays of a multi-array substrate may larger than the distance between neighboring features within a single array of the multi-array substrate. In certain embodiments, the distance between neighboring arrays of a multi-array substrate may be at least 1 mm, e.g., about 2 to about 10 mm.

The term “organelle genome” is used to refer to the genomic DNA found within an organelle. The genome of an organelle is found in the nucleic acids isolated from a cell containing that organelle. Organelles may be isolated from a cell using a variety of methods, including density and affinity methods.

An “intergenic region” of a genome is a region is between adjacent genes of that genome, where a gene includes a transcribed region. As used herein, any contiguous promoter and downstream regions required for transcription of the transcribed region is considered part of an intergenic region which is associated with a gene. An untranslated sequence which is transcribed is not considered an “intergenic region” as defined herein.

The “identity” of an organism indicates the name of a sub-population of organisms to which that organism belongs. The identity of an organism may be, for example, the genus, species, ethnic race, familial group, or given name of the organism.

A “subject” is a multicellular organism. In certain embodiments, a subject may be plant (e.g., monocot or dicot), or animal (e.g., fish, reptile, bird, insect or mammal). In one embodiment, a subject may be a human being.

A “data set” is a collection of data points obtained from reading an array. In certain embodiments, a data set may be in the form of a table of numerical information indicating the intensities of optically detectable signals produced by hybridized features.

### DETAILED DESCRIPTION

A comparative genome hybridization method is provided. In certain embodiments, the method comprises: a) contacting a first array of surface-bound nucleic acid probes with: i) a first labeled population of target nucleic acids from a genome of an organelle of a first cell; and ii) a second labeled population of target nucleic acids from a genome of an organelle of a second cell; and b) detecting binding between the surface-bound nucleic acid probes and the first and second labeled populations of target nucleic acids. Compositions for use in the subject methods are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. Thus, for example, reference to “an organelle” includes a plurality of such organelles, and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Organelle Comparative Genome Hybridization

In general terms, the subject comparative genome hybridization methods involve distinguishably labeling a first organelle genome sample and a second organelle genome sample (e.g., a first and second mitochondrial genome sample or a first and second plastid genome sample) to make two distinguishably labeled populations of target nucleic acids, contacting the labeled populations of target nucleic acids with an array of surface bound nucleic acid probes under specific hybridization conditions, and then reading the array.

The organelle genome samples employed in the subject method may contain genomic DNA (e.g., mitochondrial or plastid genomic DNA) or an amplified version thereof (e.g., organelle DNA amplified using methods similar to those of Lage et al, Genome Res. 2003 13: 294-307, published patent application US20040241658, or a polymerase chain reaction-based method, LM-PCR, or an multiple strand displacement amplification technique, for example) from an organelle of a eukaryotic cell. In certain embodiments, the first and second organelle genome samples may be both mitochondrial genome samples and may be the same as each other or different from each other. In other embodiments, the first and second organelle genome samples may be both plastid genome samples and may be the same as each other or different from each other. A plastid genome sample may contain the DNA of a chloroplast, chromoplast, leucoplast, amyloplast, elaioplast, proplastid or etioplast, for example, or any combination thereof. If the organelle genome samples are plastid genome samples, the first and second samples may be made from same type or different types of plastid. For example, the first plastid genome sample may be a chloroplast genome sample and the second plastid genome sample may be, e.g., a chloroplast, chromoplast or amyloplast genome sample.

Methods for making organelle genome samples are routine (see, e.g., Singh et al, Anal. Biochem. 1995 225:155-7; Singh et al, Anal. Biochem. 1995 225:152-5; Wolstenholme et al, Proc. Natl. Acad. Sci. 1968 61:245-52; DeSalle et al, Methods Enzymol. 1993 224:176-204; Jansen et al, Methods Enzymol. 2005 395:348-84; Hewlett et al, Methods Enzymol. 1986 118:201-12 and Ma et al, Mol. Cell Biochem. 1985 65:181-8) and, as such, need not be described any further. In certain embodiments, organelle DNA may be separated from nuclear DNA prior to performing the subject methods. However, in certain embodiments, organelle DNA may not be separated from nuclear DNA and a whole cell DNA sample may be employed. As will be described in greater detail below and in particular embodiments, a nuclear genome sample and an organelle genome sample may be produced from the same cellular composition. In these embodiments, a cellular composition may be divided into portions. A first portion of the cellular composition may be used to produce a nuclear genome sample and a second portion of the cellular composition may be used to produce an organelle genome sample, for example. Kits for isolating organelles from cells are available from a variety of manufacturers, including Pierce (Rockford, Ill.), Sigma-Aldrich (St. Louis, Mo.) and others.

An organelle genome sample may contain organelle DNA from any eukaryotic cell, e.g., an animal (fish, insect, or mammal, for example), plant (Arabidopsis, tobacco or maize, for example), protist (algae, for example) or fungal cell. In exemplary embodiments, the eukaryotic cell may be a mammalian cell such a human, mouse, rat or monkey cell. The cells used to produce an organelle genome sample may be cultured cells, cells obtained from a subject (e.g., cells of a clinical sample, e.g., a tissue biopsy, scrape or lavage or cells of a plant) or cells of a forensic sample, or for example.

As noted above, in certain embodiments, two organelle genome samples are differentially labeled and contacted with the same nucleic acid array. The different organelle genome samples may include a test sample, i.e., a sample of interest, and a reference sample to which the test sample may be compared. In certain embodiments, the different organelle genome samples are made from pairs of cell types, one cell type being a cell type of interest, e.g., an abnormal cell, and the other a control, e.g., normal, cell. Exemplary cell type pairs include, for example, cells isolated from a tissue biopsy (e.g., from a tissue having a phenotype of an organelle-related condition, from a tissue having a disease, or from an otherwise normal tissue, etc.) and normal cells from the same tissue (e.g., muscle tissue) from the same patient; cells grown in tissue culture that are immortal (e.g., cells with a proliferative mutation or an immortalizing transgene) or are primary cell lines, infected with a pathogen, or treated (e.g., with environmental or chemical agents such as a peptide, hormone, altered temperature, growth condition, compound, drug, physical stress, cellular transformation, etc.), and a normal cell (e.g., a cell that is otherwise identical to the experimental cell except that it is not immortal, infected, or treated, etc.); a cell isolated from a mammal having an organelle-related disease or condition, and a cell from a mammal of the same species (or from the same family), that is healthy; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

The organelle genome samples are distinguishably labeled using any convenient method such as, for example, primer, extension, random-priming, nick translation, etc. (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). The samples may be labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato, Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Bostan, Mass.) and POPRO3 and TOPRO3 (Molecular Probes, Eugene, Oreg.). Further suitable distinguishable detectable labels may be found in Kricka et al. (Ann Clin Biochem. 39:114-29, 2002).

The labeling reactions produce a first and second population of labeled target nucleic acids that correspond to the test and reference organelle genome samples, respectively. After nucleic acid purification and any optional pre-hybridization steps to suppress repetitive sequences (e.g., hybridization with Cot-1 DNA), the populations of labeled nucleic acids are contacted to an array of surface bound polynucleotides, as discussed above, under conditions such that nucleic acid hybridization to the surface bound polynucleotides can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C.

The labeled target nucleic acids can be contacted to the same array serially, or, in other embodiments, simultaneously (i.e., the labeled nucleic acids are mixed prior to their contacting with the surface-bound polynucleotides).

Standard hybridization techniques (using high stringency hybridization conditions) are used to probe a nucleic acid array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (Plenum Press, New York 1985). See also U.S. Pat. Nos: 6,335,167; 6,197,501; 5,830,645; and 5,665,549; the disclosures of which are herein incorporate by reference.

Following hybridization, the array-surface bound nucleic acid probes may be washed to remove unbound labeled target nucleic acids. Washing may be performed using any convenient washing protocol, where the washing conditions are typically stringent, as described above.

Following hybridization and washing, as described above, the hybridization of the labeled nucleic acids to the targets is then detected using standard techniques so that the surface of immobilized targets, e.g., the array, is read. Reading of the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent applications: Ser. No. 09/846125 “Reading Multi-Featured Arrays” by Dorsel et al.; and U.S. Pat. No. 6,406,849, which references are incorporated herein by reference.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In certain embodiments, the methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

Accordingly, in certain embodiments a pair of organelle genome samples are labeled to make two populations of differentially labeled nucleic acids, the nucleic acids are contacted with an array of surface-bound polynucleotides, and the level of labeled nucleic acids bound to each surface-bound polynucleotide is assessed.

In certain embodiments, a surface-bound nucleic acid probe is assessed by determining the level of binding of the population of labeled nucleic acids to that polynucleotide. The term “level of binding” means any assessment of binding (e.g. a quantitative or qualitative, relative or absolute assessment) usually done, as is known in the art, by detecting signal (i.e., pixel brightness) from the label associated with the labeled nucleic acids. Since the level of binding of labeled nucleic acid to a surface-bound polynucleotide is proportional to the level of bound label, the level of binding of labeled nucleic acid is usually determined by assessing the amount of label associated with the surface-bound polynucleotide.

In certain embodiments, a surface-bound nucleic acid probe may be assessed by evaluating its binding to two populations of target nucleic acids that are distinguishably labeled. In these embodiments, for a single surface-bound nucleic acid probe of interest, the results obtained from hybridization with a first population of labeled target nucleic acids may be compared to results obtained from hybridization with the second population of target nucleic acids, usually after normalization of the data. The results may be expressed using any convenient means, e.g., as a number or numerical ratio, etc.

By “normalization” is meant that data corresponding to the two populations of nucleic acids are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl:496-501, 2002, Bilban et al Curr Issues Mol Biol. 4:57-64, 2002, Finkelstein et al, Plant Mol Biol.48(1-2):119-31, 2002, and Hegde et al, Biotechniques. 29:548-554, 2000). Specific examples of normalization suitable for use in the subject methods include linear normalization methods, non-linear normalization methods, e.g., using lowess local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using the data obtained from a support-bound polynucleotide for a chromosome of known concentration in any of the chromosome compositions.

Accordingly, binding of a surface-bound nucleic acid probe to a labeled population of target nucleic acids may be assessed. In some embodiments, the assessment provides a numerical assessment of binding, and that numeral may correspond to an absolute level of binding, a relative level of binding, or a qualitative (e.g., presence or absence) or a quantitative level of binding. Accordingly, a binding assessment may be expressed as a ratio, whole number, or any fraction thereof.

Arrays

Arrays employed in comparative genome hybridization assays (CGH assays) contain an array of nucleic acid probes immobilized on a substrate, e.g., a solid support. Array platforms for performing array-based comparative genome hybridization methods are known in the art (e.g., see Pinkel et al., Nat. Genet. (1998) 20:207-211; Hodgson et al., Nat. Genet. (2001) 29:459-464; Wilhelm et al., Cancer Res. (2002) 62: 957-960). In certain aspects, CGH arrays contain a plurality (i.e., at least about 100, at least about 500, at least about 1000, at least about 2000, at least about 5000, at least about 10,000, at least about 20,000, usually up to about 100,000 or more) of addressable features each containing a nucleic acid probe that is linked to a substrate, e.g., a planar substrate. Features on an array employed in the subject methods contain nucleic acid probes that hybridize with, i.e., bind to, organelle genome sequences. Accordingly, the arrays used in the subject methods can contain plurality of different cDNAs, oligonucleotides, or inserts from phage or plasmids, etc., that are addressably arrayed. The CGH arrays employed herein may contain surface bound nucleic acid probes that are about 10-200 bases in length, about 201-5000 bases in length, about 5001-50,000 bases in length, or about 50,001-200,000 bases in length, depending on the platform used. In certain embodiments, the subject features contain oligonucleotides that are about 10 to about 200 bases, however, in certain embodiments, the oligonucleotides may be about 10 to about 100 bases, about 10 to about 80 bases, about 10 to about 50 bases, or about 10 to about 30 bases in length. In particular embodiments, the subject features contain oligonucleotides are 20-60 bases in length.

The nucleic acid probes present in a subject array are designed for detecting regions of an organelle genome, e.g., a mitochondrial genome or plastid genome. Such nucleic acid probes have a sequence that is at least partially complementary to or the same as and will base-pair with, a region of an organelle genome. Accordingly, a subject array contains a plurality of nucleic acid probes that have a sequence that is present in an organelle genome and may be used to detect a particular sequence. Such nucleic acid probes are organelle-specific in that they correspond to and may be used to detect regions of an organelle genome, even if other nucleic acids (e.g., nuclear DNA) are present. In certain embodiments, at least about 30% (e.g., at least about 50%, at least about 70%, at least about 80%, up to 100%) of the features on a subject array are for detecting regions of an organelle genome.

The nucleic acid probes contained in the subject features may be designed according to one or more particular parameters to be suitable for use in a given application, where representative parameters include, but are not limited to: length, melting temperature (TM), non-homology with other regions of the genome, hybridization signal intensities, kinetic properties under hybridization conditions, etc., see e.g., U.S. Pat. No. 6,251,588, the disclosure of which is herein incorporated by reference. In certain embodiments, the entire length of the subject nucleic acid probe, e.g., oligonucleotide probe, is employed in hybridizing to a region in a genome of interest, while in other embodiments, only a portion of the subject oligonucleotide has sequence that hybridizes to sequence found in a genome of interest, e.g., where a portion of the subject nucleic acid probe serves as a tether. For example, a given nucleic acid probe may include a 30 nt long genome specific sequence linked to a 30 nt tether, such that the nucleic acid is a 60-mer of which only a portion, e.g., 30 nt long, is genome specific.

In certain embodiments, the nucleic acid probes of a subject array may be designed to detect differences between two organelle genomes (e.g., nucleotide or polynucleotide substitutions, deletions, and insertions) of any size (i.e., at least one base pair (bp)). In particular embodiments, the polynucleotides of a subject array may be designed to detect differences between organelle genomes, where a difference (e.g., the substitution, deletion, and/or insertion) involves more than 1 basepair (e.g., involves 2 or more basepairs, 3 or more basepairs, about 5 or more basepairs, about 10 or more basepairs, about 20 or more basepairs, about 50 or more basepairs, about 100 or more basepairs, about 500 or more basepairs or more than 1 kbp). Differences in two organelle genomes, can be spontaneous or somatic (Holt et al, 1988 Nature 331:717-719; Lestienne et al, 1988), maternally inherited (Ballinger et al, 1994 Nature Genetics 7:458-459; Ballinger et al, 1992 Nature Genetics 1:11-15), or Mendelianly inherited due to predisposing nuclear mutations (Cormier et al, 1991 American Journal of Human Genetics 48:643-648; Zeviani et al, 1990. American Journal of Human Genetics 47:904-914). In a particular embodiment, the subject methods may be employed to detect a re-arrangement (e.g., an inversion of a genomic region), if the junctions of the re-arrangement are know. Exemplary re-arrangements that may be detected by the instant methods are set forth in Tables 1-4.

In certain embodiments, the nucleic acid probes of the array may be designed to detect particular differences, where a particular difference may be associated with a particular phenotype, e.g., a condition or the like. In other embodiments, the nucleic acid probes of the array may be arbitrarily chosen. In one embodiment, a subject array may contain nucleic acid probes for detecting any one or more (e.g., at least 10, at least 100 or more) of the differences in the mitochondrial genome set forth in any of Tables 1-4. The differences listed in Tables 1-4 are known in the art. All nucleotide locations in Tables 1-4 are in accordance with the human mitochondrial reference sequence of Anderson et al (Nature 1981 290:457-65; deposited as GenBank Accession No. J01415.0; GI No. 337188). The differences described in Tables 1-4 are described by the size of any insertion or deletion, the nucleotides at the junction, the nature and size of any flanking repeat, and the locations of the repeats, if present.

TABLE 1
mitochondrial genome deletions

| Deletion Junctions | Deletion | |
|---|---|---|
| (nt:nt) | Size | Repeat Location (nt) |
| 105:112 | −6 | — |
| 470:5152 | −4681 | 471-483/5152-5163 |
| 502:5443 | −4939 | 504-512/5443-5451 |
| 547:4443 | −3895 | 536-548/4430-4442 |
| 1836:5447 | −3610 | 1837-1840/5447-5451 |
| 3173:14161 | −10987 | 3166-3173/14154-14161 |
| 3323:3588 | −264 | 3330-3334/3597-3601 |
| 4398:14822 | −10422 | 4389-4398/14812-12821 |
| 5786:13923 | −8136 | — |
| 5793:12767 | −6973 | 5786-5792/12757-12763 |
| 5835:12661 | −6825 | 5836-5843/12666-12673 |
| 6023:14424 | −8400 | 6023/14423 |
| 6074:9179 | −3104 | 6080-6083/9181-9184 |
| 6075:13799 | −7723 | 6076-6084/13799-13807 |
| 6226:13456 | −7279 | 6218-6226/13447-13455 |
| 6238:14103 | −7864 | 6235-6238/14099-14102 |
| 6325:13989 | −7663 | 6326-6341/13889-14004 |
| 6329:13994 | −7664 | 6325-6340/13989-14005 |
| 6330:13994 | −7663 | 6331-6341/13994-14004 |
| 6380:14096 | −7715 | 6369-6374/14099-14104 |
| 6465:14135 | −7669 | 6466-6476/14135-14145 |
| 7193:14596 | −7402 | 7169-7173/14609-14613 |
| 7438:13476 | −6037 | 7439/13476 |
| 7449:15926 | −8476 | — |
| 7491:11004 | −3512 | 7484-7491/10996-11003 |
| 7493:12762 | −5268 | 7491-7493/12759-12761 |
| 7501:14428 | −6926 | 7502-7506/14428-14432 |
| 7635:15440 | −7804 | 7627-7630/15432-15435 |
| 7669:15437 | −7767 | 7662-7669/15429-15436 |
| 7697:12364 | −4666 | — |
| 7775:13532 | −5756 | 7769-7775/13525-13531 |
| 7777:13794 | −6016 | 7780-7783/13796-13799 |
| 7808:14799 | −6990 | 7809-7814/14799-14804 |
| 7815:15381 | −7565 | 7816-7823/15381-15388 |
| 7817:14536 | −6718 | — |
| 7829:14135 | −6305 | 7824-7829/14129-14134 |
| 7841:13905 | −6063 | 7842-7849/13905-13912 |
| 7845:9748 | −1902 | 7838-7840/9747-9749 |
| 7959:13787 | −5827 | 7954-7959/13781-13786 |
| 7974:15496 | −7521 | 7975-7982/15496-15503 |
| 7992:15730 | −7737 | 7986-7992/15723-15729 |
| 8030:16075 | −8044 | 8030-8034/16071-16075 |
| 8032:16075 | −8042 | 8030-8034/16071-16075 |
| 8034:16076 | −8041 | 8030-8034/16071-16075 |
| 8210:15339 | −7128 | — |
| 8213:13991 | −5777 | 8214-8220/13991-13997 |
| 8213:16070 | −7856 | 8207-8212/16071-16076 |
| 8271:8281 | −9 | NR |
| 8278:13770 | −5491 | 8272-8277/13763-13768 |
| 8304:15055 | −6750 | 8305-8312/15049-15056 |
| 8412:15664 | −7251 | 8407-8420/15658-15671 |
| 8426:12894 | −4467 | 8416-8420/12890-12894 |
| 8439:16075 | −7635 | 8433-8439/16068-16074 |
| 8468:13446 | −4977 | 8470-8482/13447-13459 |
| 8469:13447 | −4977 | 8470-8482/13447-13459 |
| 8476:14812 | −6335 | 8470-8476/14805-14811 |
| 8482:13460 | −4977 | 8470-8482/13447-13459 |
| 8517:15421 | −6903 | 8512-8517/15408-15412 |
| 8563:13758 | −5196 | — |
| 8563:14596 | −6032 | 8558-8563/14588-14593 |
| 8570:13236 | −4665 | 8566-8571/13237-13242 |
| 8573:15727 | −7153 | 8574-8587/15727-15737 |
| 8580:15731 | −7150 | 8581-8587/15731-15737 |
| 8582:15957 | −7374 | — |
| 8623:15662 | −7038 | 8617-8623/15656-15662 |
| 8624:13886 | −5261 | 8617-8624/13877-13885 |
| 8631:13513 | −4881 | 8625-8631/13506-13512 |
| 8631:13580 | −4949 | — |
| 8637:16084 | −7446 | — |
| 8648:16085 | −7436 | 8637-8648/16073-16084 |
| 8707:13723 | −5015 | — |
| 8823:15855 | −7031 | 8814-8823/15845-15854 |
| 8828:14896 | −6067 | 8829-8838/14896-14095 |
| 8992:16072 | −7079 | — |
| 9144:13816 | −4671 | 9137-9144/13808-13815 |
| 9180:14281 | −5100 | 9172-9175/14289-14292 |
| 9191:12909 | −3717 | 9189-9191/12906-12908 |
| 9238:15576 | −6377 | 9226-9230/15568-15572 |
| 9320:14273 | −4952 | — |
| 9357:13865 | −4507 | 9345-9361/13851-13868 |
| 9515:13055 | −3539 | 9517-9525/13055-13063 |
| 9574:12972 | −3397 | 9568-9574/12965-12971 |
| 9995:15897 | −5901 | 9998-10006/15895-15903 |
| 10050:15076 | −5025 | 10051-10055/15076-15080 |
| 10058:14593 | −4534 | 10059-10063/14593-14597 |
| 10154:15945 | −5790 | 10138-10144/15944-15950 |
| 10169:14435 | −4265 | 10161-10165/14424-14428 |
| 10190:13753 | −3562 | 10191-10198/13753-13760 |
| 10367:12829 | −2461 | 10365-10367/12826-12828 |
| 10370:15570 | −5199 | 10366-10370/15568-15572 |
| 10587:15913 | −5325 | 10576-10581/15905-15910 |
| 10598:13206 | −2607 | 10587-10592/13192-13197 |
| 10665:14856 | −4190 | 10666-10676/14856-14866 |
| 10676:14868 | −4191 | 10666-10676/14856-14866 |
| 10744:14124 | −3379 | 10745-10754/14124-14133 |
| 10941:15362 | −4420 | 10942-10951/15362-15371 |
| 10952:15837 | −4884 | 10953-10961/15837-15845 |
| 10961:15846 | −4884 | — |
| 11232:13980 | −2747 | 11234-11242/13981-13989 |
| 11368:15786 | −4417 | 11364-11368/15783-15787 |
| 12102:14412 | −2309 | 12103-12112/14412-14421 |
| 12103:14414 | −2310 | 12103-12112/14412-14421 |
| 12113:14422 | −2308 | 12103-12112/14412-14421 |
| 12203:15355 | −3151 | — |
| 14786:14791 | −4 | 14783-14786 |
| 10758:14138 | −3379 | 10151-10758/14128-14136 |
| 10925:14152 | −3226 | 10921-10925/14146-14150 |
| 11480:16071 | −4591 | NR |
| 13761:14252 | −490 | 13754-13761/14243-14250 |
| 547:4443 | −3895 | 536-548/4430-4442 |
| 5786:13923 | −8136 | 5785-5788/13918-13921 |
| 5787:13920 | −8132 | 5785-5788/13918-13921 |
| 6341:14005 | −7663 | NR |
| 7469:16071 | −8601 | 7469-7472/16071-16074 |
| 7619:15032 | −7412 | 7620-7623/15032-15035 |
| 7775:13532 | −5756 | 7769-7775/13525-13531 |
| 7794:16072 | −8277 | 7794-7799/16072-16077 |
| 7817:16071 | −8253 | 7819-7824/16071-16076 |
| 7818:16071 | −8252 | 7819-7824/16071-16076 |
| 7819:16070 | −8250 | 7819-7824/16071-16076 |
| 7819:16071 | −8251 | 7820-7825/16071-16076 |
| 7845:15761 | −7915 | 7841-7845/15751-15753 |
| 7869:16075 | −8205 | 7872-7878/16072-16078 |
| 7899:15642 | −7742 | 7905-7908/15646-15649 |
| 7914:16070 | −8155 | 7915-7918/16069-16072 |
| 7919:16070 | −8150 | 7915-7918/16069-16072 |
| 7920:16071 | −8150 | 7919-7922/16065-16068 |
| 7920:16078 | −8157 | 7915-7918/16068-16071 |
| 7922:16074 | −8151 | 7919-7922/16065-16068 |
| 7931:15761 | −7829 | 7924-7931/15760-15767 |
| 7959:13787 | −5827 | 7954-7959/13781-13786 |
| 7967:11312 | −3344 | 7968-7972/11312-11316 |
| 7989:16071 | −8081 | 7986-7990/16065-16069 |
| 7989:16071 | −8081 | 7986-8990/16065-16069 |
| 7990:16068 | −8077 | 7986-7990/16065-16069 |
| 7991:16070 | −8078 | 7986-7990/16065-16069 |
| 7992:15730 | −7737 | 7986-7992/15723-15729 |
| 8029:16071 | −8041 | 8030-8036/16071-16076 |
| 8030:16070 | −8039 | 8030-8036/16071-16076 |
| 8030:16071 | −8040 | 8030-8036/16071-16076 |
| 8030:16075 | −8044 | 8030-8034/16071-16075 |
| 8034:16076 | −8041 | 8030-8034/16071-16075 |
| 8213:16070 | −7856 | 8207-8212/16071-16076 |
| 8254:16070 | −7815 | 8252-8254/16071-16073 |
| 8316:15802 | −7485 | |
| 8344:15633 | −7288 | |
| 8387:16071 | −7683 | |
| 8439:16075 | −7635 | 8433-8439/16068-16074 |
| 8469:13447 | 4977 | 8470-8482/13447-13459 |
| 8476:14812 | −6335 | 8470-8476/14805-14811 |
| 8482:13460 | −4977 | 8470-8482/13447-13459 |
| 8553:15585 | −7031 | |
| 8580:15731 | −7150 | 8581-8587/15731-15737 |
| 8612:16072 | −7459 | |
| 8631:13513 | −4881 | 8625-8631/13506-13512 |
| 8636:16073 | −7436 | 8637-8648/16073-16084 |
| 8648:16085 | −7436 | 8637-8648/16073-16084 |
| 8727:13698 | −4970 | 8712-8717/13688-13693 |
| 8774:13990 | −5215 | 8772-8777/14001-14006 |
| 8931:13766 | −4834 | 8931-8936/13768-13773 |
| 9079:15312 | −6232 | 9075-9079/15307-15311 |
| 9193:15322 | −6128 | 9189-9194/15319-15325 |
| 9281:13810 | −4528 | 9274-9281/13801-13808 |
| 9440:14083 | −4642 | 9431-9440/14072-14081 |
| 9444:13964 | −4519 | 9437-9444/13955-13962 |
| 9497:13735 | −4237 | 9486-9497/13722-13733 |
| 9532:14135 | −4602 | 9526-9532/14127-14133 |
| 9574:1395 | −4376 | 9571-9574/13946-13949 |
| 9587:13953 | −4366 | 9579-9587/13943-13951 |
| 9591:13052 | −3460 | 9546-9591/13046-13051 |
| 9600:14138 | −453 | 9596-9600/14132-14136 |
| 9609:14147 | −4537 | 9606-9610/14141-14145 |
| 9844:16064 | −6219 | 9833-9844/16061-16070 |
| 9966:15801 | −5834 | 9952-9956/15798-15802 |

TABLE 2
mitochondrial genome insertions

| Insert size (bp) | Insert (nt) | Insertion location |
|---|---|---|
| 4 | CCCC | 8277 MT-CO2/MT-TK |
| | | (Intergenic region) |
| +˜5 | CCCCC | 956-965 MT-RNR1 |
| +˜12 | CCCCCCCCCCCC | 5895-5899 MT-TY/MT-CO1 |
| | | (Intergenic region) |
| 266 | 308-573 D-Loop | 573 D-Loop region |
| | region | |
| +˜200 | 302-493 D-Loop | 493 D-Loop region |
| | region | |

TABLE 3
mitochondrial genome rearrangements

| Parental | Insert size | | | |
|---|---|---|---|---|
| molecule | (bp) | | mtDNA Species | Insert(nt) |
| Complete | +7.6 | kb | 24.2 kb, 16.6 kb, | Deleted mtDNA |
| (16.6 kb) | | | 15.2 kb, 7.6 kb | fragment |
| | | | | 15056-6130 |
| Complete | +10.6 | kb | 26 kb, 16.6 kb, | Deleted mtDNA |
| (16.6 kb) | | | 10 kb | fragment |
| | | | | with junction |
| | | | | 8718:15239 |
| Complete | +6.4 | kb | 21 kb, 16.6 kb, | Deleted mtDNA |
| (16.6 kb) | | | 6 kb | fragment |
| | | | | 13445-3318 |
| Complete | +6.1 | kb | 16.6 kb, 12 kb, | Deleted mtDNA |
| (16.6 kb) | | | 6.1 kb | fragment |
| | | | | 14822-4398 |
| Complete | +4.6 | kb | 21.2 kb, 16.6 kb | Deleted mtDNA |
| (16.6 kb) | | | | fragment |
| | | | | with junction |
| | | | | 3567:15537 |
| Complete | +4.6 | kb | 25.8 kb, 21.2 kb, | Deleted mtDNA |
| (16.6 kb) | | | 16.6 kb | fragment(s) |
| | | | | with junction |
| | | | | 3567:15537 |
| Complete | +4 kb, +8 kb | | 24.6 kb, 20.6 kb, | Duplicated 4 |
| (16.6 kb) | | | 16.6 kb, 4 kb | kb mtDNA |
| | | | | fragment(s) |
| | | | | with junction |
| | | | | ˜3270:˜16070 |
| Deleted | +12.4 | kb | 24 kb, 16.6 kb, | Deleted mtDNA |
| (12.4 kb) | | | 11 kb | fragment |
| | | | | with junction |
| | | | | 10676:14868 |
| Deleted | +12.4 | kb | 24 kb, 16.6 kb, | Deleted mtDNA |
| (12.4 kb) | | | 11 kb | fragment |
| | | | | with junction |
| | | | | 10665:14856 |
| Deleted | +10.2 | kb | 20 kb, 16.6 kb, | Deleted mtDNA |
| (10.2 kb) | | | 10 kb | fragment |
| | | | | with junction |
| | | | | 9328:15576 |
| Deleted | +9.2 | kb | 21.2 kb, 16.6 kb, | Two 4.6 KB |
| (12 kb) | | | 4.6 kb | deleted mtDNA |
| | | | | fragments |
| | | | | with junction |
| | | | | 3577:15547 |

TABLE 4
somatic mutations

| Position | mutation | |
|---|---|---|
| 584:4442 | 3857 | bp del |
| 3232:3236 | TAAG-T | |
| 3323:3588 | 264 | bp del |
| 4398:14822 | 10422 | bp del |
| 5788:16071 | 10282 | bp del |
| 6173:16078 | 9904 | bp del |
| 8469:13447 | 4977 | bp del |
| 8723:9215 | 491 | bp del |
| 9436:15913 | 6476 | bp del |

At present, the entire sequences of approximately 778 mitochondria and 49 plastids are readily accessible via NCBI's Genbank database. Further information on the mitochondrial genome, particularly that of the human mitochondrial genome, including annotation information, gene function information, diseases and conditions associated with genome alterations and further genome alterations including single nucleotide polymorphisms, is found in the MITOMAP database of Brandon et al, 2005 (MITOMAP: a human mitochondrial genome database—2004 update. Nucleic Acids Research 33:D611-613, 2005), as well as Wallace et al, 1995 (Report of the committee on human mitochondrial DNA. In Cuticchia A J (ed) Human gene mapping 1995: a compendium. Johns Hopkins University Press, Baltimore, pp 910-954). The MITOMAP database may be accessed via the world wide website of the MITOMAP organization.

In certain embodiments, the above-described array may be present on a substrate that contains multiple arrays, i.e., a “multi-array substrate”. In addition to the above-described organelle array, the substrate may contain a further array of nucleic acid probes for detecting regions of a nuclear genome or, in other embodiments, a further array of nucleic acid probes for detecting mRNA transcribed from a nuclear genome. Accordingly, in one embodiment, a multi-array substrate containing: a) a first array of surface-bound nucleic acid probes that bind to regions of an organelle genome of a cell; and b) a second array of surface-bound polynucleotide probes for detecting regions of a nuclear genome of the cell is provided. In another embodiment, a multi-array substrate containing: a) a first array of surface-bound nucleic acid probes that bind to regions of an organelle genome of a cell; and b) a second array of surface-bound polynucleotide probes for detecting mRNA of the cell is provided. In these embodiments, the substrate may contain at least two, e.g., four, six, 8, 12 or 16 or more arrays. In certain embodiments, the first and second arrays may be spatially separated. Arrays for detecting regions of a nuclear genome and arrays for detecting nuclear gene expression products are well known in the art (see, e.g., Pinkel et al., Nat. Genet. (1998) 20:207-211; Hodgson et al., Nat. Genet. (2001) 29:459-464; Wilhelm et al., Cancer Res. (2002) 62: 957-960, U.S. Pat. Nos. 6,465,182, 6,335,167, 6,251,601, 6,210,878, 6,197,501, 6,159,685, 5,965,362, 5,830,645, 5,665,549, 5,447,841 and 5,348,855; and published U.S. patent application 20040191813).

Utility

The methods described above, in one embodiment, may be employed to assess the relative abundance of organelle DNA in two different organelle genome samples, which, in turn, provides an indication of the relative copy number of the organelle genome in the cells used to obtain those samples. In another embodiment, the subject methods may be employed to assess the relative abundance of a sequence in two different organelle genome samples. In another embodiment, the methods may be employed to detect specific DNA sequences in an organelle genome.

The above-described methods find use in a variety of diagnostic and research methods. In particular, the above-described methods may be employed to diagnose, or investigate an organelle-related condition. Mitochondria-related conditions include a variety of muscle-related conditions (e.g., myopathies), hearing-related conditions and vision-related conditions and include, but not are not limited to: Leber hereditary optic neuropathy, mitochondrial myopathy, Alzheimer's disease, lethal infantile mitochondrial myopathy, Parkinsons's disease, maternal myopathy, mardiomyopathy, neurogenic muscle weakness, ataxia, retinitis pigmentosa; Leigh disease, fatal infantile cardiomyopathy, mitochondrial encephalomyopathy, lactic acidosis, stroke, dystonia, myoclonic epilepsy, ragged red muscle fibers, maternally inherited hypertrophic cardiomyopathy, chronic progressive external ophthalmoplegia, Kearns-Sayre syndrome, diabetes mellitus, Type 2 Diabetes, deafness (which can be maternally inherited or aminoglycoside-induced), chronic intestinal pseudo-obstruction, ophthalmoplegia, progressive encephalopathy, sensorineural hearing loss, adult-onset dystonia, prostate cancer, myoglobinuria, exercise intolerance, therapy-resistant epilepsy, rhabdomyolysis, acquired idiopathic sideroblastic anemia, amylotrophic lateral schlerosis, septo-optic dysplasia, multiple sclerosis, Parson syndrome, stroke, autism, and migraine. Many of the mitochondrial-related conditions listed above are associated with particular nucleotide changes in the mitochondrial genome. Many exemplary nucleotide changes that are associated with and are thought to be a factor in producing the above-recited phenotypes are listed in Tables 1-4. In these embodiments, a cellular sample may be obtained from a subject and the sample tested using the methods described above.

The above-described methods may be further employed to investigate the effect of an environmental stimulus (e.g., a physical stimulus or a chemical such as a drug or a pesticide) on either the overall copy number or the copy number of regions of an organelle genome. In these methods, a cell (either in vitro or in vivo) may be exposed to a stimulus (e.g., contacted with a drug or exposed to light, heat, or cold, for example), and tested using the methods described above. Exemplary chemicals that may be employed in the above-described methods include small organic or inorganic molecules (i.e., molecules having a molecular weight of 5-1000 Da, 100-750 Da, 200-500 Da, or less than 500 Da in size), polysaccharides, polynucleotides, polypeptides, etc.

The effects of known drugs, particularly drugs such as antibiotics or drugs that are know to inhibit nucleic acid replication (e.g., nucleotide or nucleoside analogs), may also be investigated. Such drugs include, but are not limited to AZT (zidovudine), ddl (didanosine), ddC (zalcitabine), d4T (stavudine), 3TC (lamivudine), vidarabine, acyclovir, gancyclovir, valganciclovir, nevirapine, delavirdine, didanosine, zalcitabine, and zidovudine.

In other embodiments, the subject methods may be employed to provide the identity of a subject or forensic sample. In these embodiments, an organelle sample is assessed using the above-described methods, and the results obtained from the methods may be, for example, compared to suitable database containing information about genomes of known identity. The identity of the subject or sample (e.g., the species of plant, for example, or, for a human individual, his name or family) may be determined if the results obtained from the methods correlate (i.e., match) with an organelle genome of known identity.

In certain embodiments, if a change in the organelle genome is detected using the above-described methods, the subject from which the target nucleic acids was made may be investigated to determine whether its cells are heteroplasmic or heteroplastic (i.e., whether a single cell of the subject contains organelles containing different genomes).

In a particular embodiment, the methods described above may be employed to identify regions (e.g., genes) in a nuclear genome whose presence or expression is required for maintenance of the mitochondrial genome in a cell. In these embodiments, a sample may be divided in at least two parts. One part of the sample will be assessed using the methods described above, and another part of the sample may be used to assess the presence of regions in the nuclear genome gene expression products in the sample. In certain embodiments, the method may include: a) assaying an organelle genome sample by comparative genome hybridization to produce a first data set; b) assaying a nuclear genome sample by comparative genome hybridization to produce a second data set; and c) comparing said first data set and said second data set. In other embodiments, the method may include: a) assaying an organelle genome sample by comparative genome hybridization to produce a first data set; b) assaying a nuclear gene expression using a gene expression array to produce a second data set; and c) comparing said first data set and said second data set. These methods may involve a) contacting a first array of surface-bound nucleic acid probes with: i) a first labeled population of target nucleic acids made from a genome of an organelle of a first cell; and ii) a second labeled population of target nucleic acid made from a genome of an organelle of a second cell; b) reading the array to detect binding between the surface-bound nucleic acid probes and the first and second labeled populations of target nucleic acids and produce a first data set; c) contacting a second array of surface-bound nucleic acid probes with: i) a third labeled population of target nucleic acids made from a nucleus of the first cell; and ii) a fourth labeled population of target nucleic acids made from a nucleus of the second cell; and d) reading the second array to detect binding between the surface-bound nucleic acid probes and the third and fourth labeled populations of target nucleic acid and produce a second data set; and e) comparing the first data set and the second data set.

In other embodiments, the method may involve a) contacting a first array of surface-bound nucleic acid probes with: i) a first labeled population of target nucleic acid made from a genome of an organelle of a first cell; and ii) a second labeled population of target nucleic acid made from a genome of an organelle of a second cell; b) reading the array to detect binding between the surface-bound nucleic acid probes and the first and second labeled populations of target nucleic acid and produce a first data set; c) contacting a second array of surface-bound nucleic acid probes with: i) a third labeled population of target nucleic acids made from mRNA of the first cell; and ii) a fourth labeled population of target nucleic acids made from mRNA of the second cell; and d) reading the second array to detect binding between the surface-bound nucleic acid probes and the third and fourth labeled populations of target nucleic acids and produce a second data set; and e) comparing the first data set and the second data set.

In one exemplary embodiment, a cellular sample containing a cell population or tissue sample is obtained from test and reference samples (e.g., from a patient, tissue or cell culture that, in certain embodiments may have altered mitochondrial genome). The cellular samples are split, and mitochondria are enriched from one fraction and nuclei enriched from the other. Standard density-based or affinity-based methods may be used to obtain the mitochondrial and nuclear fraction. In an alternative embodiment, nuclei and mitochondria can be isolated from a single cellular sample using density-based or affinity-based methods. The mitochondrial and nuclear genomic DNA from each of the test and reference samples may be optionally amplified using random primers or another known method, and labeled. The test and reference mitochondrial genome samples may be labeled using spectrally distinguishable labels (e.g., Cy3 and Cy5). Likewise the test and reference nuclear genome samples may be labeled using spectrally distinguishable labels (e.g., Cy3 and Cy5). The labeled mitochondrial genome samples are contacted with an array containing polynucleotides that bind to the mitochondrial genome and the labeled nuclear genome samples are contacted with a spatially distinct array containing polynucleotides that bind to the nuclear genome. The arrays are read to produce data, and the data used to evaluate a) the relative copy number of mitochondrial genome sequence in the cellular samples, b) the relative copy number of specific sequences in the mitochondrial genome in the cellular samples, and c) compare changes in mitochondrial genome copy number to changes in the copy number of nuclear regions. Further, depending on the test sample, the data may be used to correlate a phenotype of the test cellular sample (e.g., a disease, drug response, environmental response or age-related phenotype) to a change in the mitochondrial genome.

Similar procedures may be employed to evaluate chloroplast copy number.

Kits

Also provided by the subject invention are kits for practicing the subject methods, as described above. The subject kits at least include an array for detecting regions of an organelle genome, as discussed above.

In certain embodiments, the array may be part of a multi-array substrate that contains an organelle array, as discussed above, and an array for detecting regions of a nuclear genome or mRNA expression products thereof. A subject kit may further include one or more additional components necessary for carrying out an array-based organelle CGH assay, such as organelle genome preparation reagents, buffers, labels, reagents for amplifying a genomic sample, and the like. The kits may also include a denaturation reagent for denaturing a sample, buffers such as hybridization buffers, wash mediums, enzyme substrates, reagents for generating a labeled sample, negative and positive controls and written instructions for using the arrays for carrying out an array based assay. Such kits also typically include instructions for use in practicing array-based assays. The various components of the kit may be present in separate containers or certain compatible components may be precombined into a single container, as desired. As such, the kits may include one or more containers such as vials or bottles, with each container containing a separate component for the assay, and reagents for carrying out an array assay such as a nucleic acid hybridization assay or the like.

The kits may also include a computer readable-medium including and instructions that may include directions for use of the invention.

The instructions of the above-described kits may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming may be recorded on a suitable recording medium.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method comprising:
a) contacting a first array of surface-bound nucleic acid probes with:
i) a first labeled population of target nucleic acids from a genome of an organelle of a first cell; and
ii) a second labeled population of target nucleic acids from a genome of an organelle of a second cell; and
b) detecting binding between said surface-bound nucleic acid probes and said first and second labeled populations of target nucleic acids.

2. The method of claim 1, wherein said method further comprises determining relative amounts of bound first labeled populations of target nucleic acids and bound second labeled populations of target nucleic acids.

3. The method of claim 1, wherein the method further comprises determining differences in numbers of copies of one or more sequences in organelle genomes of said first and second cells.

4. The method of claim 2, wherein the method further comprises determining the relative expression of one or more nuclear encoded gene products in said first and second cells.

5. The method of claim 2, wherein determining relative expression further comprises determining differences in numbers of copies of one or more sequences in nuclear genomes of said first and second cells.

6. The method of claim 5, wherein said method employs a multi-array substrate.

7. The method of claim 2, wherein the method further comprises correlating relative amounts determined with a condition of an organism from which the first or second cell was obtained.

8. The method of claim 7, wherein the condition is a mitochondrial-related disease.

9. The method of claim 8, wherein the condition is a muscle-related, hearing-related or vision-related disorder.

10. The method of dependent claim 2, wherein the method further comprises correlating relative amounts determined with an amount of heteroplasmy.

11. The method of claim 1, wherein said first cell and said second cell are obtained from different tissues of the same patient and said method evaluates differences in numbers of copies of one or more sequences in organelle genomes of said different tissues.

12. The method of claim 1, wherein said first cell and said second cell are obtained from the same patient at different times and said method evaluates differences in numbers of copies of one or more sequences in organelle genomes at said different times.

13. The method of claim 1, wherein at least one of said first and second cells is exposed to a stimulus and said method evaluates differences in numbers of copies of one or more sequences in organelle genomes in response to said stimulus.

14. The method of claim 13, wherein said stimulus is a chemical compound.

15. The method of claim 14, wherein said chemical compound is a drug.

16. The method of claim 13, wherein said stimulus is an environmental condition.

17. The method of claim 2, wherein said relative amounts are employed to evaluate the identity of an organism from which the first or second cell was obtained.

18. The method of claim 1, wherein said surface-bound nucleic acid probes are for detecting a plurality of different organelle genome regions.

19. The method of claim 1, wherein said first and second labeled populations of target nucleic acids are distinguishably labeled.

20. The method of claim 1, wherein said first and second cells are cultured cells or cells obtained from a subject.

21. The method of claim 1, wherein said method identifies deletions or insertions in said genome of said organelle of said first cell relative to said genome of said organelle of said second cell.

22. The method of claim 1, wherein said detecting produces a first data set.

23. The method of claim 22, wherein said data set is stored.

24. The method of claim 1, further comprising:
c) contacting a second array of surface-bound nucleic acid probes with:
i) a third labeled population of target nucleic acids from a nuclear genome of said first cell;
i) a fourth labeled population of target nucleic acid from a nuclear genome of said second cell; and
d) detecting binding between said surface-bound polynucleotide probes and said third and fourth labeled populations of polynucleotides.

25. The method of claim 24, wherein said first array and said second array are on the same substrate.

26. The method of claim 24, wherein said first and said second array are on different substrates

27. The method of claim 24, wherein said detecting produces a second data set.

28. The method of 1, wherein said organelle is a chloroplast or mitochondria.

29. A method comprising:
a) assaying an organelle genome sample by comparative genome hybridization to produce a first data set;
b) assaying a nuclear genome sample by comparative genome hybridization to produce a second data set; and
c) comparing said first data set and said second data set.

30. A multi-array substrate comprising:
a) a first array of surface-bound nucleic acid probes that bind to regions of an organelle genome of a cell; and
b) a second array of surface-bound nucleic acid probes that bind to regions of a nuclear genome of said cell,
wherein said first array and said second array are spatially separated.

31. The multi-array substrate of claim 30, wherein said surface-bound nucleic acid probes are oligonucleotide probes.

32. The multi-array substrate of claim 30, wherein said first array comprises surface-bound nucleic acid probes for detecting intergenic regions of said organelle genome.

33. The multi-array substrate of claim 30, wherein said organelle genome is a mitochondrial genome and said cell is an animal cell.

34. The multi-array substrate of claim 30, wherein said organelle genome is a chloroplast genome and said cell is a plant cell.

35. A kit comprising the multi-array substrate of claim 1 and reagents for obtaining genomic DNA from an organelle of a eukaryotic cell.

36. The kit of claim 35, further comprising reagents for labeling said genomic DNA.
